(19) United States
(12) Patent Application Publication
(10) Pub. No.: US 2024/0253041 A1
(43) Pub. Date: Aug. 1, 2024

(54) SORTING DEVICE AND METHOD BASED ON ELECTRIC SPARK CAVITATION BUBBLES

(71) Applicant: TSINGHUA UNIVERSITY, Beijing (CN)

(72) Inventors: Zheng YOU, Beijing (CN); Zeheng JIAO, Beijing (CN); Jingjing ZHAO, Beijing (CN); Yong HAN, Beijing (CN)

(21) Appl. No.: 18/567,169

(22) PCT Filed: Dec. 15, 2021
(86) PCT No.: PCT/CN2021/138421

§ 371 Date: Dec. 5, 2023

(30) Foreign Application Priority Data
Nov. 26, 2021 (CN) 202111421030.6
Dec. 13, 2021 (CN) 202111522984.6

(51) Int. Cl. B01L 3/00

(52) U.S. Cl. CPC B01L 3/502761; B01L 2200/0652; B01L 2300/0645; B01L 2300/0663; B01L 2300/0864; B01L 2300/087; B01L 2400/046

(57) ABSTRACT

A sorting device based on an electric spark cavitation bubble includes: a liquid flow subsystem including a sheath flow channel, a sample flow channel, and a main flow channel, where the main flow channel is divided into a waste flow channel and a collection flow channel via a bifurcation port; a detecting subsystem configured to collect a pulse signal excited by the cell sample, and convert the pulse signal into an electrical signal; a data acquiring and processing subsystem configured to acquire and analyze the electrical signal, and issue a sorting instruction based on an analysis result; and a cavitation bubble generating subsystem configured to generate the cavitation bubble according to the sorting instruction, where the cavitation bubble pushes a liquid to generate a jet, and the bifurcation port is located within a range corresponding to the jet nozzle.

### CROSS-REFERENCE TO RELATED APPLICATIONS

This application is a national stage entry under 35 U.S.C. § 371 of International Application No. PCT/CN2021/138421, filed on Dec. 15, 2021, which claims priority to Chinese Patent Application No. 202111522984.6, filed on Dec. 13, 2021, and Chinese Patent Application No. 202111421030.6, filed on Nov. 26, 2021. The entire disclosures of the above-identified applications are incorporated herein by reference.

### TECHNICAL FIELD

The present disclosure is generally applied to flow cytometers, relates to the field of flow sorting, and particularly to a sorting device and method based on an electric spark cavitation bubble.

### BACKGROUND

Flow cytometer is an instrument used for high-throughput, multi-parameter analysis and sorting of biological particles such as cells. Flow cytometers are classified into two types: an analytical type and a sorting type. A common commercial flow cytometer uses a high-voltage electrostatic field to deflect charged droplets that wrap cells in an open space to achieve single-cell sorting. With the high-voltage electrostatic field, the flow cytometer has a fast sorting speed, but it has a large volume and may have other problems of aerosol contamination and cross-contamination. The mechanical impact during the sorting process will affect the cell activity. Therefore, the sorting system of the commercial flow cytometer needs to be improved, and there is an urgent need for developing a sorting means with a simple structure, a low cost and capable of solving the aerosol contamination and the cross-contamination.

Electrical spark cavitation bubble sorting is a sorting method that uses a high-voltage pulse to discharge between positive and negative electrodes to generate plasma to heat the liquid to generate cavitation bubbles, so as to realize the sorting of cells. The electrical spark cavitation bubble sorting has the advantages of low cost and completely enclosed sorting, but there are still some problems such as poor durability of electrode materials and insufficient success rate of sorting.

### SUMMARY

According to a first aspect of embodiments of the present disclosure, there is provided a sorting device based on an electric spark cavitation bubble, including: a liquid flow subsystem including a sheath flow channel, a sample flow channel, and a main flow channel, in which a cell sample forms a single-cell axial flow after passing through the sample flow channel and enters the main flow channel, and the main flow channel is divided into a waste flow channel and a collection flow channel via a bifurcation port at a downstream position: a detecting subsystem configured to collect a pulse signal excited by the cell sample, and convert the pulse signal into an electrical signal; a data acquiring and processing subsystem configured to acquire and analyze the electrical signal, and issue a sorting instruction based on an analysis result; and a cavitation bubble generating subsystem configured to generate the cavitation bubble according to the sorting instruction, in which the cavitation bubble is used to push a liquid to generate a jet, the jet is sprayed from a jet nozzle to act on the cell sample, and the bifurcation port is located within a range corresponding to the jet nozzle.

According to a second aspect of embodiments of the present disclosure, there is provided a sorting method based on an electric spark cavitation bubble, including the following steps: (1) labeling a target cell: (2) introducing the sheath liquid and a cell sample solution from the sheath flow channel and the sample flow channel respectively, focusing the cell sample solution wrapped by the sheath liquid into the single-cell axial flow; and introducing the single-cell axial flow into the main flow channel: (3) exciting an optical signal as the single-cell axial flow flows through a detection region of the detecting subsystem, receiving the optical signal by a photoelectric detecting unit to form the pulse signal, and converting, by the detecting subsystem, the pulse signal into an electrical signal; and (4) acquiring, by the data acquiring and processing subsystem, the electrical signal and determining whether the cell sample solution is the target cell based on a preset condition: in response to determining that the cell sample solution is the target cell, issuing, by the data acquiring and processing subsystem, a sorting instruction, and generating the cavitation bubble by the cavitation bubble generating subsystem, in which the cavitation bubble is used to push the target cell into the collection flow channel; and in response to determining that the cell sample solution is not the target cell, not issuing the sorting instruction, and the cell sample solution flowing enter the waste flow channel.

### BRIEF DESCRIPTION OF THE DRAWINGS

These and other aspects and advantages of embodiments of the present disclosure will become apparent and more readily appreciated from the following descriptions made with reference to the accompanying drawings, in which:

FIG. 1 is a schematic diagram illustrating a structure of a sorting device of the present disclosure;

FIG. 2 is a schematic diagram illustrating a relative position of a bifurcation port and a jet nozzle of the present disclosure:

FIG. 3 shows images illustrating a sorting effect of the present disclosure where a bifurcation port is located at different positions:

FIG. 4 is a schematic diagram illustrating a sorting principle of the present disclosure:

FIG. 5 shows images of a positive electrode after multiple discharges when the positive electrode of the present disclosure is metal tungsten:

FIG. 6 shows images of a positive electrode after multiple discharges when the positive electrode of the present disclosure is metal platinum:

FIG. 7 is a schematic diagram illustrating an implementation of dynamically adjusting a discharge time interval in the present disclosure; and

FIG. 8 is a block diagram illustrating a sorting control method of the present disclosure.

### REFERENCE NUMERAL

sheath flow channel 1, sample flow channel 2, negative electrode 3, positive electrode 4, waste flow channel 5, collection flow channel 6, waste tube 7, collection tube 8, high-voltage discharge circuit 9, laser 10, detecting subsystem 11, data acquiring and processing subsystem 12, cavitation bubble 13, non-target cell 14, target cell 15, jet nozzle 16, cavitation chamber 17, main flow channel 18, first movement direction 19, second movement direction 20, third movement direction 21, dichroic mirror 22, pulse signal 23, bifurcation port 24, jet nozzle range 25, filter 26, detection channel 27 for forward scattered light, detection channel 28 for fluorescence, detection channel 29 for side scattered light, single-cell axial flow 30.

### DETAILED DESCRIPTION

Embodiments of the present disclosure are described in detail below; and examples of the embodiments are shown in the drawings. The same or similar elements and the elements having same or similar functions are denoted by like reference numerals throughout the descriptions. The embodiments described herein with reference to drawings are explanatory, and used to generally understand the present disclosure. The embodiments shall not be construed to limit the present disclosure.

In order to solve the above-mentioned technical problems of the poor durability of the electrode material and the low sorting success rate of the electrical spark cavitation bubble sorting existing in the related art, the present disclosure provides a sorting device and method based on an electric spark cavitation bubble.

The technical solution used in the present disclosure for solving the above-mentioned technical problems is as follows.

On the one hand, the present disclosure provides a sorting device based on an electric spark cavitation bubble, including: a liquid flow subsystem including a sheath flow channel, a sample flow channel, and a main flow channel, in which a cell sample forms a single-cell axial flow after passing through the sample flow channel and enters the main flow channel, and the main flow channel is divided into a waste flow channel and a collection flow channel via a bifurcation port at a downstream position: a detecting subsystem configured to collect a pulse signal excited by the cell sample, and convert the pulse signal into an electrical signal: a data acquiring and processing subsystem configured to acquire and analyze the electrical signal, and issue a sorting instruction based on an analysis result; and a cavitation bubble generating subsystem configured to generate the cavitation bubble, in which the cavitation bubble is used to push a liquid to generate a jet, the jet is sprayed from a jet nozzle to act on the cell sample, and the bifurcation port is located within a range corresponding to the jet nozzle.

In some embodiments, the cavitation bubble generating subsystem includes a high-voltage discharge circuit, a volume of the cavitation bubble is controlled by adjusting a discharge time of the high-voltage discharge circuit, and the discharge time is dynamically adjusted based on a time interval between a current discharge pulse and several preceding discharge pulses.

In some embodiments, the cavitation bubble generating subsystem further includes: a positive electrode, a negative electrode, and a cavitation chamber. The cavitation chamber is located at a side of the main flow channel, the cavitation chamber is in communication with the main flow channel through the jet nozzle.

In some embodiments, a material of the positive electrode is platinum or tungsten, and a material of the negative electrode is platinum, tungsten or stainless steel.

In some embodiments, the detecting subsystem includes a laser, a detection channel for forward scattered light, a detection channel for side scattered light and several detection channels for fluorescence.

In some embodiments, an optical signal is generated after the cell sample is irradiated with laser light emitted by the laser, and the optical signal is divided according to a fluorescence band and is received by a photoelectric detecting unit to form the pulse signal.

In some embodiments, the optical signal includes a scattered light signal and a fluorescence signal.

In some embodiments, the cell sample flowing out from the waste flow channel enters a waste tube, and the cell sample flowing out from the collection flow channel enters a collection tube.

In some embodiments, the sheath flow channel is configured to pass a sheath liquid, and the sample flow channel is configured to pass the cell sample.

On the other hand, the present disclosure provides a sorting method based on an electric spark cavitation bubble, including the following steps: (1) labeling a target cell: (2) introducing the sheath liquid and a cell sample solution from the sheath flow channel and the sample flow channel respectively, focusing the cell sample solution wrapped by the sheath liquid into the single-cell axial flow; and introducing the single-cell axial flow into the main flow channel: (3) exciting an optical signal as the single-cell axial flow flows through a detection region of the detecting subsystem, receiving the optical signal by a photoelectric detecting unit to form the pulse signal, and converting, by the detecting subsystem, the pulse signal into an electrical signal; and (4) acquiring, by the data acquiring and processing subsystem, the electrical signal and determining whether the cell sample solution is the target cell based on a preset condition: when the cell sample solution is the target cell, issuing, by the data acquiring and processing subsystem, a sorting instruction, and generating the cavitation bubble by the cavitation bubble generating subsystem, in which the cavitation bubble is used to push the target cell into the collection flow channel; and when the cell sample solution is not the target cell, not issuing the sorting instruction, and the cell sample solution flowing enter the waste flow channel.

Compared with related art, the present disclosure achieves the following technical effects. In the present disclosure, platinum or tungsten is used as the material of the positive electrode, and platinum, tungsten or stainless steel is used as the material of the negative electrode, thus improving the durability of the electrode material. In the present disclosure, the bifurcation port is located within the range corresponding to the jet nozzle, thus avoiding the sorting failure caused by the rebound phenomenon. In the present disclosure, the volume of the cavitation bubble is controlled by adjusting the discharge time of the high-voltage discharge circuit, and the discharge time is dynamically adjusted based on a time interval between a current discharge pulse and several preceding discharge pulses, thus effectively controlling the volume of the cavitation bubble, obtaining uniform-sized cavitation bubbles, and ensuring the stability of the system. Therefore, the present disclosure can realize the low-cost, high-speed and high-precision cell sorting.

The sorting device and method based on an electric spark cavitation bubble provided according to the embodiments of the present disclosure will be described below with reference to the accompanying drawings.

As shown in FIGS. 1-8, the present disclosure provides a sorting device based on an electric spark cavitation bubble, including: a liquid flow subsystem, a detecting subsystem 11, a data acquiring and processing subsystem 12 and a cavitation bubble generating subsystem.

The liquid flow subsystem includes a sheath flow channel 1, a sample flow channel 2 and a main flow channel 18, in which the sheath flow channel 1 and the sample flow channel 2 are all communicated with the main flow channel 18, and the sheath flow channel 1 and the sample flow channel 2 are located at an upstream position. It can be understood that the sheath flow channel 1 is configured to pass a sheath liquid, and the sample flow channel 2 is configured to pass a cell sample. The sheath liquid is configured to wrap a cell sample solution. Under the joint action of the sheath liquid and the cell sample solution, the cell sample forms a single-cell axial flow 30 and enters the main flow channel 18, and cells in the sample forming the single-cell axial flow 30 flow into the main flow channel 18 one by one.

It can be understood that, according to the present disclosure, structures of the sheath flow channel 1 and the sample flow channel 2 are not limited, and those skilled in the art can design a required structure according to the actual situation, as long as the cell sample can form the single-cell axial flow 30 and enter the main flow channel 18.

The main flow channel 18 is divided into a waste flow channel 5 and a collection flow channel 6 via a bifurcation port 24 at a downstream position. It can be understood that the waste flow channel 5 is configured to pass non-target cell(s) 14, and the collection flow channel 6 is configured to pass target cell(s) 15.

In some embodiments, the cell sample flowing out from the waste flow channel 5 enters a waste tube 7, and the cell sample flowing out from the collection flow channel 6 enters a collection tube 8. That is, the waste flow channel 5 and the collection flow channel 6 correspond to the waste tube 7 and the collection tube 8, respectively.

It can be understood that a microfluidic chip can be used to replace a common flow chamber, and it may further improve the integration of the sorting device. The microfluidic chip includes a chip substrate and flow channels arranged on the substrate.

When the single-cell axial flow 30 flows through the main flow channel 18, it will pass through a detection region of a detection subsystem 11. The detection subsystem 11 includes a laser 10, a detection channel 27 for forward scattered light, a detection channel 29 for side scattered light and several detection channels 28 for fluorescence. When the cell sample passes through the detection region, the laser 10 emits a laser light to the cell sample, and the cell sample is excited to generate an optical signal, and the optical signal includes a scattered light signal and a fluorescence signal. In the detection subsystem 11, an optical path is formed with a dichroic mirror 22 and a filter 26, and the scattered light signal and the fluorescence signal are separated according to the fluorescence band after passing through the optical path, and are received by a photoelectric detection unit of the detection subsystem 11 to form a pulse signal 23. The detection subsystem 11 converts the pulse signal 23 into an electrical signal and transmits it to the data acquiring and processing subsystem 12.

The data acquiring and processing subsystem 12 is configured to acquire and analyze the electrical signal, and issue a sorting instruction based on an analysis result. In some embodiments, before the cells to be sorted enter the main flow channel 18, the cells to be sorted are labeled and modified with a label such as a fluorescent antibody. When the target cell 15 and the non-target cell 14 pass through the detection region of the detection subsystem 11, the pulse signals 23 generated by the excitation of the cells are different, and thus the electrical signals converted by the different pulse signals 23 are different. The data acquiring and processing subsystem 12 acquires and analyzes the electrical signals, and determines whether they are target cells 15 or not according to a preset condition.

It can be understood that, after the analysis and determination of the data acquiring and processing subsystem 12, if it is a target cell 15, a sorting instruction is issued to control the cavitation bubble generating subsystem to generate the cavitation bubble 13, and the cavitation bubble 13 pushes the target cell 15 into the collection flow channel 6; and if it is a non-target cell 14, no sorting instruction is issued, thus allowing the cell sample solution to enter the waste flow channel 5.

The cavitation bubble generating subsystem is configured to generate the cavitation bubble(s) 13. The cavitation bubble generating subsystem includes a positive electrode 4, a negative electrode 3, and a cavitation chamber 17, and a high-voltage discharge circuit 9. When the data acquiring and processing subsystem 12 issues a sorting instruction, the high-voltage circuit discharges. Once the high-voltage circuit discharges, the cavitation bubble 13 is generated at a tip of the positive electrode 4, and the cavitation bubble 13 is first formed in the cavitation chamber 17, and then pushes a liquid to generate a jet. The jet is sprayed via a jet nozzle 16 to act on the cell sample 15, to make the target cell 15 enter the collection flow channel 6. The cavitation chamber 17 is located at a side of the main flow channel 18, and the cavitation chamber 17 is in communication with the main flow channel 18 through the jet nozzle 16. A single energy consumption for generating the cavitation bubble 13 is at a level from 10 μJ to 10 mJ, and a diameter of the cavitation bubble 13 is from 50 μm to 200 μm.

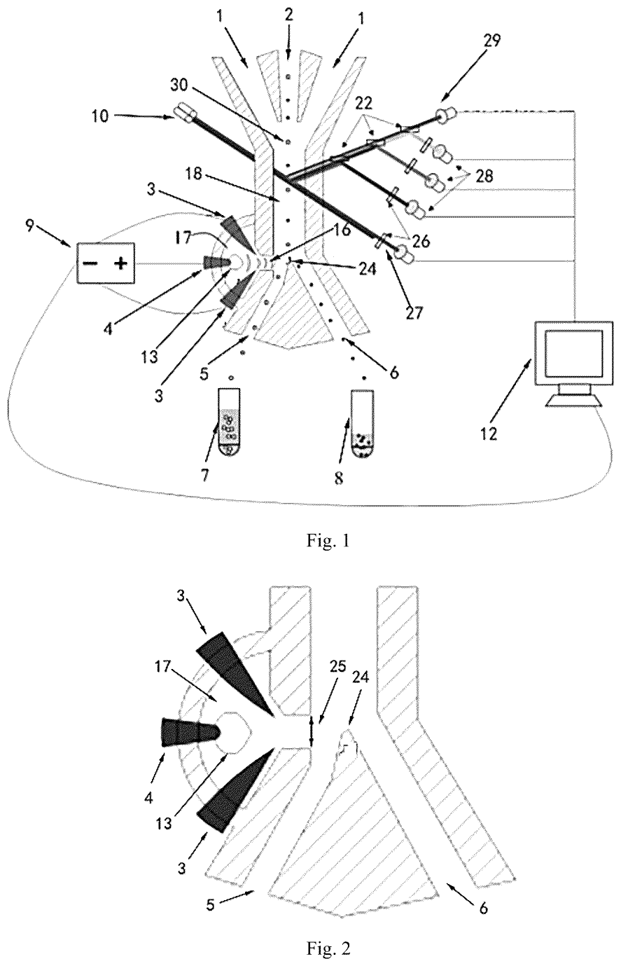

The bifurcation port 24 is located within a jet nozzle range 25. As shown in FIG. 2, a tip of the bifurcation port 24 is located within the range corresponding to the jet nozzle 16. In some embodiments, the tip of the bifurcation port 24 is located at a central axis of the jet nozzle range 25.

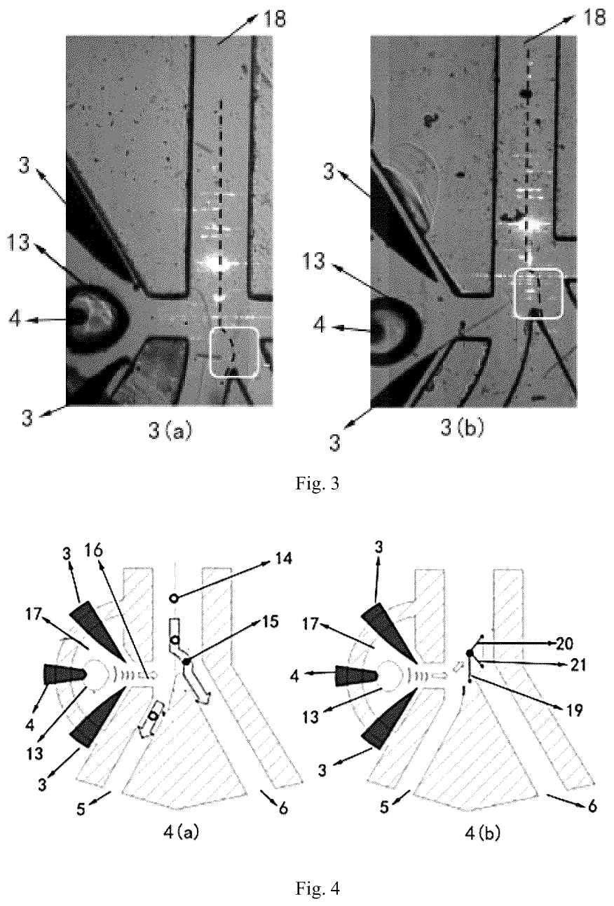

The sorting effects of the bifurcation port 24 at different positions is shown in FIG. 3. FIG. 3(a) shows a trajectory of a cell particle movement in a sorting structure of a first design, and FIG. 3(b) shows a trajectory of a cell particle movement in a sorting structure of the present disclosure. The first design is that the bifurcation port 24 is located downstream of the jet nozzle 16, but the design of the present disclosure is moving the bifurcation port 24 upwards, that is, the position of the bifurcation port 24 is set at the central axis of the jet nozzle range 25. As shown in FIG. 3(a), it can be seen from a part marked by a square block that when sorting with the first design structure, a rebound phenomenon may occur, that is, the cell particles are pushed laterally by the jet generated by the cavitation bubble 13, but returns to the center of the flow channel after the push. In this design, the bifurcation port 24 is far away from the jet nozzle 16, after the sorting, the cell particles laterally return to the initial position eventually, resulting in the failure of the sorting. The trajectory of the cells is shown by a dotted line drawn in FIG. 3(a). In the sorting structure designed in the present disclosure, the position of the bifurcation port 24 is moved upwards, and it is arranged at a position directly corresponding to the jet nozzle 16, as shown in FIG. 3(b). In the design of the present disclosure, the jet will generate an oblique backward push force, and this push force, together with a push force of the liquid flow in the main flow channel 18, acts on the cell particles to cause them to move laterally: After the cell particles are pushed by the jet, they enter the collection flow channel 6 faster, so as to avoid the sorting failure caused by the rebound phenomenon. As shown in FIG. 3(b), it can be seen from a part marked by a square block that there is no rebound phenomenon in the design of the present disclosure, and the trajectory of cells is shown by the dotted line drawn in FIG. 3(b).

A principle for the sorting is described with reference to FIG. 4. As shown in FIG. 4(a), a solid circle indicates the target cell 15, a hollow circle indicates the non-target cell 14, and the target cell 15 and the non-target cell 14 in the form of the single-cell axial flow 30 flow in the main flow 20) channel 18. When the target cell 15 is close to the bifurcation port 24, the discharge between the positive electrode 4 and the negative electrode 3 is triggered, the cavitation bubble 13 is generated at the tip of the positive electrode 4, and the cavitation jet is further generated. The cavitation jet sprayed from the jet nozzle 16 pushes the target cell 15 to deviate from the original trajectory. As shown in FIG. 4(b), the push force of the jet to the target cell 15 causes the target cell 15 to produce a second movement direction 20, and a direction after combination of the second movement direction 20 and an original, first movement direction 19 of the target cell 15 is a movement direction of the sorted target cell 15, namely a third movement direction 21. That is, after the sorting, the target cell 15 moves along the third movement direction 21 and smoothly enters the collection flow channel 6. As shown in FIG. 4(a), the position of the bifurcation port 24 is within the range corresponding to the jet nozzle, which can effectively prevent the target cell 15 from rebounding during collapse of the cavitation bubble 13.

A material of the positive electrode is platinum or tungsten, and a material of the negative electrode is platinum, tungsten or stainless steel. The positive electrode 4 is a consumable material, and since the cavitation bubble 13 is generated at the tip of the positive electrode 4, long-term cavitation discharge will lead to a certain material loss. The electric spark discharge is accompanied by ablation of the positive electrode 4, the metal material is peeled off, and the tip of the positive electrode 4 will be worn out. With the ablation loss of the tip of the positive electrode 4, a distance between the positive electrode and the negative electrode gradually increases, reducing a discharge stability of the system.

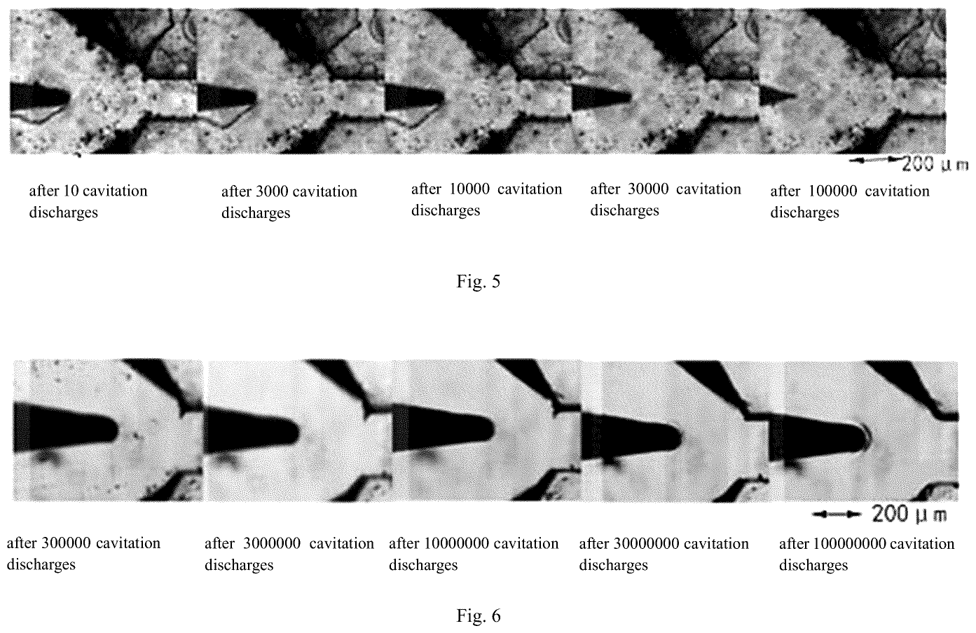

In the case where both the material of the positive electrode and the material of the negative electrode are metal tungsten, images after multiple discharges are shown in FIG. 5. FIG. 5 shows images after 10, 3000, 10000, 30000 and 100000 cavitation discharges of the tungsten electrode respectively, which are taken by a high-speed camera. It can be seen from FIG. 5 that the tungsten electrode has ablation and peeling phenomenon after 30000 cavitation discharges. In this image, the distance between the positive electrode and the negative electrode is increased significantly, and the discharge stability of the system decreases.

In the case where the material of the positive electrode is metal platinum and the material of the negative electrode is metal tungsten, images after multiple discharges are shown in FIG. 6. FIG. 6 shows images after 30000, 300000, 1000000, 3000000, and 10000000 cavitation discharges of the tungsten electrode respectively, which are taken by a high-speed camera. It can be seen from FIG. 6 that the performance of the platinum electrode is stable, and there is no obvious loss after 1e7 cavitation discharges (the actual measurement can reach 1e8 discharges). In practical applications, the materials of the positive and negative electrodes can be flexibly selected according to requirements of cost and sorting times.

In flow detection, an interval between particles passing through the detection region is accidental. In the process of detecting and sorting a large number of particles, the time interval between two adjacent particles may be too short, resulting in excessive volume of the cavitation bubble 13. In order to overcome the impact of high-speed and random discharge, avoid a great change in the volume of the cavitation bubble 13, and ensure the stability of the system, it is found that there is a linear relationship between the volume of the cavitation bubble 13 and the discharge energy in a certain range, and the discharge energy can be controlled by controlling the discharge duration, thereby effectively regulating the volume of the cavitation bubble 13.

In the present disclosure, the volume of the cavitation bubble 13 is controlled by adjusting a discharge time of the high-voltage discharge circuit 9, and the discharge time is dynamically adjusted according to a time interval between a current discharge pulse and several preceding discharge pulses. In the present disclosure, dynamically adjusting the discharge duration adjusts a single discharge duration by a program algorithm so as to maintain the size of the cavitation bubble 13.

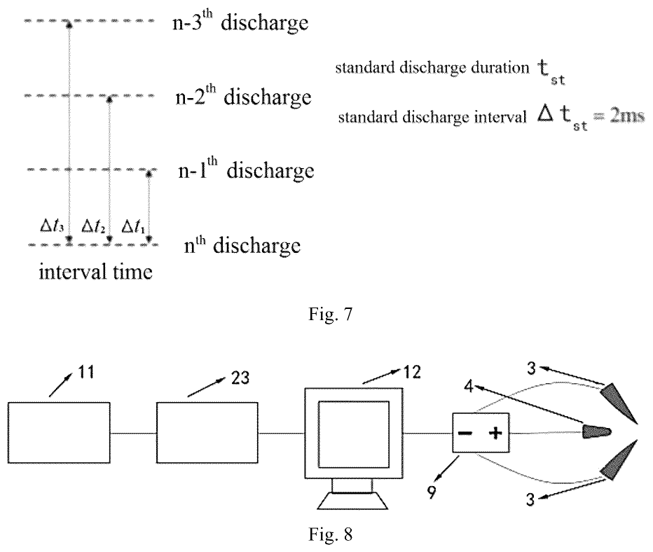

The present disclosure provides in an embodiment dynamically adjusting the discharge duration as shown in FIG. 7. According to studies, under the same buffer flow rate, the higher the frequency of the generation of the cavitation bubble 13 is, the shorter the time interval is, and the more the volume of the bubble increases. On this basis, a dynamic pulse duration sorting is designed.

As shown in FIG. 7, assuming that the current sorting is an n^{th} discharge, a discharge duration of the n^{th} sorting is calculated with reference to intervals between the current sorting and the three previous (n−1)^{th}, (n−2)^{th} and (n−3)^{th} sorting, to calibrate the discharge duration tₙ of the current sorting to control the volume of the generated cavitation bubble 13. The discharge duration tₙ is corrected and calculated by Formula 1 with respect to a standard discharge duration tₛₜ:

$${t}_{n} = {t}_{st} [ 1 - {k}_{1} ( 1 - \frac{Δ {t}_{1}}{Δ {t}_{st}} ) \cdot H ( 1 - \frac{Δ {t}_{1}}{Δ {t}_{st}} ) - {k}_{2} ( 2 - \frac{Δ {t}_{2}}{Δ {t}_{st}} ) \cdot H ( 2 - \frac{Δ {t}_{2}}{Δ {t}_{st}} ) - {k}_{3} ( 3 - \frac{Δ {t}_{3}}{Δ {t}_{st}} ) \cdot H ( 3 - \frac{Δ {t}_{3}}{Δ {t}_{st}} ) ] , \qquad Formula 1$$

where Δtₛₜ is a standard discharge interval of 2 ms, and k₁, k₂, k₃ are correction coefficients determined by experiments. When the sorting interval of adjacent target cells 15 is greater than Δtₛₜ, it is considered that the current discharge is not affected by the previous discharge bubbles. When the adjacent target time interval is less than Δtₛₜ, dynamic adjustment needs to be performed.

Further, H=H(x), the expression of H(x) is shown in Formula 2:

$$H ( x ) = \{ \begin{matrix}1 & x \geq 0 \\ 0 & x < 0\end{matrix} . \qquad Formula 2$$

The data acquiring and processing subsystem 12 performs the algorithm process of dynamically adjusting the discharge duration, and the data acquiring and processing subsystem 12 includes a computer or a microprocessor (MCU) with a computing power, or a field programmable gate array (FPGA), which can determine the interval time of the target cell 15, and realize the function of fast real-time calculation of the discharge time, and output the sorting signal.

The sorting method based on the electric spark cavitation bubble includes the following steps: (1) labeling the target cell 15: (2) introducing the sheath liquid and the cell sample solution from the sheath flow channel 1 and the sample flow channel 2, respectively, focusing the cell sample solution wrapped by the sheath liquid into the single-cell axial flow 30, and introducing the single-cell axial flow 30 into the main flow channel 18: (3) exciting the optical signal as the single-cell axial flow 30 flows through the detection region of the detecting subsystem 11, receiving the optical signal by the photoelectric detecting unit to form the pulse signal 23, and converting, by the detecting subsystem 11, the pulse signal 23 into the electrical signal; and (4) acquiring, by the data acquiring and processing subsystem 12, the electrical signal and determining whether the cell is the target cell 15 based on a preset condition, in response to determining that the cell is the target cell 15, issuing the sorting instruction, generating the cavitation bubble 13 by the cavitation bubble generating subsystem, and the cavitation bubble 13 pushing the target cell 15 into the collection flow channel 6; and in response to determining that the cell is the non-target cell 14, not issuing the sorting instruction, and the cell sample solution flowing enter the waste flow channel 5.

Reference throughout this specification to “an embodiment,” “some embodiments.” “an example,” “a specific example,” or “some examples,” means that a particular feature, structure, material, or characteristic described in connection with the embodiment or example is included in at least one embodiment or example of the present disclosure. The appearances of the above phrases in various places throughout this specification are not necessarily referring to the same embodiment or example of the present disclosure. Furthermore, the particular features, structures, materials, or characteristics may be combined in any suitable manner in one or more embodiments or examples. In addition, different embodiments or examples and features of different embodiments or examples described in the specification may be combined by those skilled in the art without mutual contradiction.

In addition, terms such as “first” and “second” are used herein for purposes of description and are not intended to indicate or imply relative importance or significance. Thus, the feature defined with “first” and “second” may include one or more this feature. In the description of the present disclosure, “a plurality of” means at least two, for example, two or three, unless specified otherwise.

Although embodiments of present disclosure have been shown and described above, it should be understood that changes, alternatives, and modifications can be made in the embodiments without departing from the principle and spirit of the present disclosure. The scope of the present disclosure is defined by the claims and their equivalents.

1. A sorting device based on an electric spark cavitation bubble, comprising:
a liquid flow subsystem comprising a sheath flow channel, a sample flow channel, and a main flow channel, wherein a cell sample forms a single-cell axial flow after passing through the sample flow channel and enters the main flow channel, and the main flow channel is divided into a waste flow channel and a collection flow channel via a bifurcation port at a downstream position;
a detecting subsystem configured to collect a pulse signal excited by the cell sample, and convert the pulse signal into an electrical signal;
a data acquiring and processing subsystem configured to acquire and analyze the electrical signal, and issue a sorting instruction based on an analysis result; and
a cavitation bubble generating subsystem configured to generate a cavitation bubble according to the sorting instruction, wherein the cavitation bubble is used to push a liquid to generate a jet, the jet is sprayed from a jet nozzle to act on the cell sample, and the bifurcation port is located within a range corresponding to the jet nozzle.

2. The device of claim 1, wherein the cavitation bubble generating subsystem comprises a high-voltage discharge circuit, a volume of the cavitation bubble is controlled by adjusting a discharge time of the high-voltage discharge circuit, and the discharge time is dynamically adjusted based on a time interval between a current discharge pulse and several preceding discharge pulses.

3. The device of claim 1, wherein the cavitation bubble generating subsystem further comprises: a positive electrode, a negative electrode, and a cavitation chamber, wherein the cavitation chamber is located at a side of the main flow channel, and the cavitation chamber is in communication with the main flow channel through the jet nozzle.

4. The device of claim 3, wherein a material of the positive electrode is platinum or tungsten, and a material of the negative electrode is platinum, tungsten or stainless steel.

5. The device of claim 1, wherein the detecting subsystem comprises a laser, a detection channel for forward scattered light, a detection channel for side scattered light and several detection channels for fluorescence.

6. The device of claim 5, wherein an optical signal is generated after the cell sample is irradiated with a laser light emitted by the laser, and the optical signal is divided according to a fluorescence band and is received by a photoelectric detecting unit to form the pulse signal.

7. The device of claim 6, wherein the optical signal comprises a scattered light signal and a fluorescence signal.

8. The device of claim 1, wherein the cell sample flowing out from the waste flow channel enters a waste tube, and the cell sample flowing out from the collection flow channel enters a collection tube.

9. The device of claim 1, wherein the sheath flow channel is configured to pass a sheath liquid, and the sample flow channel is configured to pass the cell sample.

10. A sorting method based on an electric spark cavitation bubble, performed by the device of claim 1, the method comprising:
(1) labeling a target cell;
(2) introducing a sheath liquid and a cell sample solution from the sheath flow channel and the sample flow channel respectively, focusing the cell sample solution wrapped by the sheath liquid into the single-cell axial flow, and introducing the single-cell axial flow into the main flow channel;
(3) exciting an optical signal as the single-cell axial flow flows through a detection region of the detecting subsystem, receiving the optical signal by a photoelectric detecting unit to form the pulse signal, and converting, by the detecting subsystem, the pulse signal into an electrical signal; and
(4) acquiring, by the data acquiring and processing subsystem, the electrical signal and determining whether the cell sample solution is the target cell based on a preset condition;
in response to determining that the cell sample solution is the target cell, issuing, by the data acquiring and processing subsystem, the sorting instruction, and generating the cavitation bubble by the cavitation bubble generating subsystem, wherein the cavitation bubble is used to push the target cell into the collection flow channel; and
in response to determining that the cell sample solution is not the target cell, not issuing the sorting instruction, and the cell sample solution flowing enter the waste flow channel.

11. The device of claim 7, wherein the scattered light signal and the fluorescence signal are separated according to the fluorescence band after passing through an optical path of the detecting subsystem, wherein the optical path is provided with a dichroic mirror and a filter.

12. The device of claim 1, wherein the waste flow channel and the collection flow channel are separated from a tip of the bifurcation port, and the tip of the bifurcation port is located at a central axis of the jet nozzle.

13. The device of claim 1, wherein a diameter of the cavitation bubble is from 50 μm to 200 μm.
